(19) United States
(12) Patent Application Publication
(10) Pub. No.: US 2022/0148715 A1
(43) Pub. Date: May 12, 2022

(54) METHOD FOR SUPPORTING WORKFLOWS IN A LABORATORY ENVIRONMENT BY MEANS OF AN ASSISTANCE SYSTEM

(71) Applicant: LabTwin GmbH, Berlin (DE)

(72) Inventors: Ferencz Sandor Paldy, Goettingen (DE); Steffen Gloth, Goettingen (DE); Magdalena Zadara, Berlin (DE); Jonas Kulessa, Berlin (DE)

(21) Appl. No.: 17/429,151

(22) PCT Filed: Feb. 6, 2020
(86) PCT No.: PCT/EP2020/053014

§ 371 Date: Aug. 6, 2021

(30) Foreign Application Priority Data
Jul. 2, 2019 (DE) 10 2019 103 078.1

(51) Int. Cl. G16H 40/20; G16H 10/60; G16H 40/67

(52) U.S. Cl. CPC G16H 40/20; G16H 40/67; G16H 10/60

(57) ABSTRACT

A method for supporting laboratory processes in a laboratory environment, in particular a bioprocess engineering laboratory environment, by an assistance system, wherein a number of laboratory entities, such as a number of laboratory devices, are associated with the laboratory environment, and wherein, by the assistance system, in a configuration step, the laboratory environment is mapped in an interchangeable laboratory data model using data technology, wherein in an interaction step, user inputs, in particular speech inputs, may be entered by the assistance system via a user interface, and predetermined user commands which have been matched with the laboratory data model are derived from the user inputs, wherein in an implementation step, the derived user commands are implemented by the assistance system, based on the laboratory data model.

### CROSS-REFERENCE TO RELATED APPLICATIONS

This application is a national stage application under 35 U.S.C. 371 of International Patent Application Ser. No. PCT/EP2020/053014, entitled “Method for Supporting Workflows in a Laboratory Environment by Means of an Assistance System,” filed Feb. 6, 2020, which claims priority from German Patent Application No. DE 10 2019 103 078.1, filed Feb. 7, 2019, the disclosure of which is incorporated herein by reference.

### FIELD OF THE TECHNOLOGY

Various embodiments relate to a method for supporting workflows in a laboratory environment by means of an assistance system, and to an assistance system for carrying out such a method.

### BACKGROUND

In today's laboratory environments, there are high requirements for accuracy, precision, and reproducibility in the execution of laboratory processes. In this regard, it is a challenge to execute laboratory processes efficiently, particularly in experimental laboratory environments for which no standardized laboratory processes are routinely defined.

In this context, various assistance systems for supporting laboratory personnel have become known, which can be summarized under the abbreviation LIMS (laboratory information and management system) or under the abbreviation ELN (electronic laboratory notebook). These are software-based data processing systems which support laboratory personnel with the provision and processing of laboratory data during a laboratory process. The known LIMS or ELN systems hardly go beyond the digitization of traditional paper-based laboratory documentation; thus, increasing efficiency when executing laboratory processes is possible only to a limited extent.

### SUMMARY

The object of various embodiments is to specify a method for supporting laboratory processes in a laboratory environment, in particular a bioprocess engineering laboratory environment, by means of an assistance system, which is accompanied by an increase in the efficiency of everyday laboratory work.

The above object is achieved by a method for supporting workflows in a laboratory environment by means of an assistance system as described herein.

Essentially, the fundamental consideration is to provide an interchangeable laboratory data model for mapping the laboratory environment using data technology, in which it is possible to use said model consistently throughout the laboratory environment and in every phase of the laboratory process. A number of laboratory entities, such as laboratory devices, are associated with the laboratory environment which is mapped by the laboratory data model.

For efficiently creating the laboratory data model, it is initially proposed that in a configuration step, the laboratory environment is mapped in the interchangeable laboratory data model using data technology, by means of the assistance system. The configuration step may be repeated regularly or at least with each change in the laboratory environment, so that an updated laboratory data model is always available.

The interchangeabilty of the laboratory data model is an important aspect of the solution according to the proposal. Due to the fact that the laboratory data model is interchangeable as such, it is readily possible to adapt the assistance system to a new laboratory environment.

As mentioned above, the laboratory data model is used in every phase of the laboratory process. In detail, it is provided that user inputs, in particular speech inputs, may be entered in an interaction step by means of the assistance system via a user interface, wherein predetermined user commands are derived from the user inputs which have been matched with the laboratory data model. What is interesting here is the fact that the derivation of the predetermined user commands depends on the laboratory data model. This takes into account the fact that equipping the laboratory environment with different laboratory devices results in different user commands being available to the laboratory user. This makes it easy to rule out the possibility of user commands occurring erroneously which do not correspond to the laboratory devices present in the laboratory environment. In this respect, the method according to the proposal is associated with a reduction in the probability of errors during the execution of the laboratory processes.

According to the proposal, it is further provided that the derived user commands are implemented in an implementation step by means of the assistance system, based on the laboratory data model. It is thus ensured that the implementation of the derived user commands is also tailored to the respective laboratory environment, which, as mentioned above, is mapped in the interchangeable laboratory data model using data technology. It is thus possible to achieve a high level of reliability and, if necessary, an optimization of the implementation of the derived user commands, in an efficient manner.

It is obvious that the continuous availability of the laboratory data model increases the aforementioned efficiency in everyday laboratory work, and in particular reduces the susceptibility to errors when executing laboratory processes. Furthermore, with the method according to the proposal, it is possible to increase user-friendliness, since the laboratory user can be supported by means of the assistance system in every phase of the laboratory process, tailored to the current laboratory environment.

In various embodiments, the assistance system according to the proposal may be implemented at least partially in a cloud-based manner, thus simplifying in particular an inter-laboratory optimization of laboratory processes. Alternatively or in addition, the assistance system may run at least partially as an app on a smart device, thus increasing user-friendliness.

Various embodiments relate to the consideration of taking into account at least one piece of state information in support of the workflows. In some embodiments, the piece of state information is a location of the laboratory user in the laboratory environment, which regularly determines which laboratory entities are relevant in the upcoming interaction and implementation steps. Alternatively or in addition, the state information may also relate to laboratory device values.

The definition of the laboratory data model for mapping the laboratory environment is described herein. The object-oriented structure of the laboratory data model as described herein can play a particular role. The object-oriented approach is not only associated with simply creating the laboratory data model in the configuration step. Rather, the data encapsulation inherent in the object-oriented approach makes it easy to reuse laboratory entities which have already been modeled, and provides a high level of security against incorrect modeling.

Various embodiments relate to the configuration step, which can be carried out based on a library of library objects. The advantages of the object-oriented structure of the laboratory data model become fully evident here.

Various embodiments ensure that the current respective laboratory data model is accessed at any time. Any minor change in the laboratory data model thus immediately affects the interaction step and the implementation step.

Various embodiments relate to making user inputs via speech inputs. Here, the use of the laboratory data model plays a very special role with respect to reducing input errors. The speech processing according to the proposal comprises, in a manner customary per se, a speech recognition step based on a language model, and a semantic analysis step based on the semantic model. Particularly noteworthy is the fact that, the language model depends on the laboratory data model, and that the semantic model correspondingly depends on the laboratory data model. This means that both the speech recognition step and the semantic analysis step may be tailored specifically to the respective laboratory environment, possibly even to the part of the laboratory environment relating to the laboratory user. In the simplest case, it is thus possible to reduce the vocabulary present in the language model and the range of commands available in the semantic model in such a way that the vocabularies and commands which are likely to be irrelevant with respect to the specific laboratory environment are not taken into consideration from the outset in the speech recognition step or in the semantic analysis step. This not only reduces the probability of errors, but also the computing power required for speech processing.

Various embodiments relate to details of the implementation step which can be carried out according to an implementation rule. In an optional variant, the respective implementation rule is contained in the associated laboratory data object, so that the implementation step may also be easily tailored to the laboratory entities of the laboratory environment.

An assistance function of the assistance system according to the proposal is the efficient creation and updating of laboratory documentation.

In some embodiments, a laboratory documentation data structure for mapping the actual laboratory process in the laboratory environment is defined, comprising a number of laboratory documentation data objects. Particularly efficient and simultaneously clear documentation results, in that at least one laboratory data object is associated with at least a portion of the laboratory documentation objects in each case. In general, the laboratory documentation data structure can be structured in an object-oriented manner, in the above manner.

Various embodiments relate to further assistance functions such as actuating laboratory entities, requesting consumables, and translating the documentation data structure. The last three assistance functions mentioned may also be implemented in a particularly targeted manner in that the laboratory data model is consistently available in its respectively current form.

By means of the availability of the laboratory data, a further possibility results for reducing the probability of errors, in that, in a plausibility step, the respective user input is checked according to a plausibility rule in terms of plausibility with respect to the laboratory environment.

According to further embodiments, the assistance system for carrying out the method according to the proposal is disclosed as such. Reference may be made to all embodiments with respect to the method according to the proposal.

Various embodiments provide a method for supporting laboratory processes in a laboratory environment, in particular a bioprocess engineering laboratory environment, by means of an assistance system, wherein a number of laboratory entities, such as a number of laboratory devices, are associated with the laboratory environment, and wherein, by means of the assistance system, in a configuration step, the laboratory environment is mapped in an interchangeable laboratory data model using data technology, wherein in an interaction step, user inputs, in particular speech inputs, may be entered by means of the assistance system via a user interface, and predetermined user commands which have been matched with the laboratory data model are derived from the user inputs, wherein in an implementation step, the derived user commands are implemented by means of the assistance system, based on the laboratory data model.

In various embodiments, the assistance system is implemented at least partially in a cloud-based manner, and/or in that the assistance system runs at least partially as an app on a smart device.

In various embodiments, the interaction step and/or the implementation step is/are also carried out as a function of at least one piece of state information, wherein the state information relates to the location of the laboratory user in the laboratory environment, and/or to the laboratory entities located in a predefined vicinity of the laboratory user, and/or to the state of an laboratory entity.

In various embodiments, in a readout step, laboratory device values, in particular measured values, of laboratory entities, in particular of laboratory devices, can be received via a device interface for determining the state of a laboratory entity by means of the assistance system.

In various embodiments, the laboratory data model comprises a number of laboratory data objects which respectively map a laboratory entity of the laboratory environment using data technology, such as the laboratory data objects are stored in the respective laboratory entity and/or can be read out of the respective laboratory entity.

In various embodiments, the laboratory data model is structured in an object-oriented manner, based on predetermined data classes of laboratory entities, in such a way that the laboratory data objects are respectively parameterized instances of a respective laboratory entity data class, such as laboratory entities representing a laboratory device are associated with a laboratory entity data class “laboratory device,” and/or in that laboratory entities representing a sample holder are associated with a laboratory entity data class “sample holder,” and/or in that the laboratory entities representing a laboratory user are associated with a laboratory entity data class “laboratory user.”

In various embodiments, an instance of the object-oriented laboratory data model is interchangeably stored as such in the assistance system.

In various embodiments, in the configuration step, the laboratory data model is compiled from a library of library objects via the user interface, such as by associating library data objects with the laboratory data model on the user side in a graphical configuration screen, for example, using drag & drop.

In various embodiments, the library objects respectively represent a laboratory entity data class.

In various embodiments, the laboratory data model is adapted, in particular continuously, to the respective current laboratory environment, and in that the interaction step and the implementation step always access the respective current laboratory data model, such as the laboratory data model is adapted to the respective current laboratory environment by means of a user input.

In various embodiments, in the interaction step, audio signals are detected via the user interface, and in that in a speech recognition step, a structured text is generated from the audio signals, based on a language model.

In various embodiments, the speech recognition step is carried out as a function of the laboratory data model and/or as a function of the state information, such as the language model underlying the speech recognition step is selected or modified as a function of the laboratory data model and/or as a function of a piece of state information, and/or in that at least a portion of the language model is included in the laboratory data model, in particular in the laboratory data objects.

In various embodiments, the interaction step comprises a semantic analysis step, in which a semantic analysis of the structured text is carried out, based on a semantic model, and in that, in the semantic analysis, the respective user command is derived from the structured text.

In various embodiments, the semantic model underlying the semantics analysis step is selected or modified as a function of the laboratory data model and/or as a function of a piece of state information, and/or in that at least a portion of the semantic model is included in the laboratory data model, in particular in the laboratory data objects.

In various embodiments, an implementation rule is provided for each user command, according to which the respective command is implemented, such as at least a portion of the implementation rule is included in the associated laboratory data object.

In various embodiments, a control sequence for implementing the user command and the laboratory entities involved in the implementation of the user command are included in the implementation rule.

In various embodiments, laboratory documentation of the actual laboratory process in the laboratory environment is defined, and in that a predetermined user command is the updating of the laboratory documentation, such as the updating of the laboratory documentation takes places in an event-based manner, further that an event triggering the updating can be a user input.

In various embodiments, a laboratory documentation data structure for mapping the actual laboratory process in the laboratory environment is associated with the laboratory documentation, and in that the laboratory documentation data structure comprises a number of laboratory documentation data objects which respectively map work progress in the laboratory process.

In various embodiments, the work progress mapped by the laboratory documentation data objects are events, in particular user-related events and/or device-related events.

In various embodiments, at least one laboratory data object is associated with at least a portion of the laboratory documentation data objects in each case.

In various embodiments, the updating of the laboratory documentation data structure is carried out based on the piece of state information.

In various embodiments, at least a portion of the received laboratory values are associated with at least a portion of the laboratory documentation data objects.

In various embodiments, a predetermined user command is the actuation of laboratory entities, in particular laboratory devices, and in that the actuation of the laboratory entities is carried out in response to a corresponding user command, in particular according to the associated implementation rule, based on the laboratory data model, in particular based on the corresponding laboratory data object.

In various embodiments, a predetermined user command is the request for consumables, and in that the request for consumables is carried out in response to a corresponding user command, in particular according to the associated implementation rule, based on the laboratory data model, in particular based on the corresponding laboratory data object.

In various embodiments, a predetermined user command comprises a translation step in which the documentation data structure is translated into a selected national language, such as into a natural language form, based on the translation rule.

In various embodiments, in a plausibility step, the user input is checked according to a plausibility rule in terms of plausibility with respect to the laboratory environment, in particular with respect to the laboratory data model, such as in the event of an implausible user input, a warning message is issued via the user interface.

Various embodiments provide an assistance system for carrying out a method as described herein.

### BRIEF DESCRIPTION OF THE DRAWINGS

Various aspects will be explained in greater detail below with reference to a drawing depicting only one exemplary embodiment. The following are shown:

FIG. 1 depicts a schematic representation of the essential method steps of a method according to the proposal;

FIG. 2 depicts the basic structure of the laboratory data model on which the method according to FIG. 1 is based;

FIG. 3 depicts a configuration screen for carrying out the configuration step of the method according to FIG. 1;

FIG. 4 depicts a schematic representation of the essential method steps of the interaction step of the method according to FIG. 1; and

FIG. 5 depicts the basic structure of a laboratory documentation data structure of the method according to FIG. 1.

### DETAILED DESCRIPTION

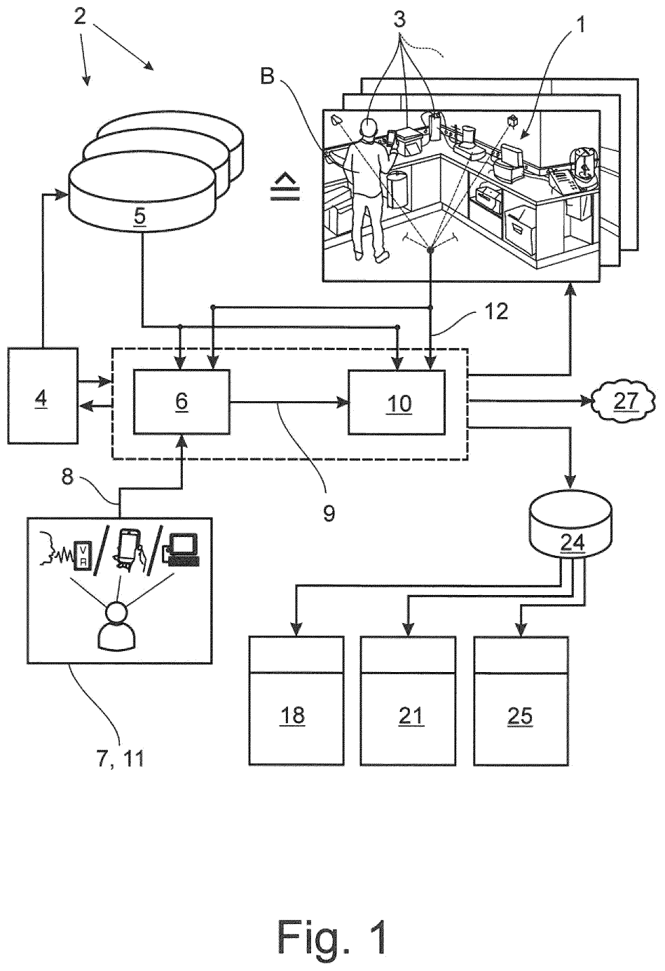

The method according to the proposal is used for supporting laboratory processes in a laboratory environment 1, here, a bioprocess engineering laboratory environment 1, by means of an assistance system 2. FIG. 1 shows that a number of laboratory entities 3 are associated with the laboratory environment 1. The laboratory entities 3 may, for example, be laboratory devices or the like, as well as laboratory personnel, as will be shown below.

For explaining details of the method according to the proposal, an exemplary laboratory process will first be presented, to which reference will be made below. With the aid of this reference laboratory process, it is possible to depict the details of the method according to the proposal in a particularly clear manner.

The reference laboratory process relates to the production of an aqueous buffer, followed by a check of the pH value. The reference laboratory process comprises the following work steps:
1. Determining the buffer properties, in particular the setpoints relating to the volume, the pH value, the solvent, the buffer salts, the salt concentration, and any additives.
2. Calculating the buffer salt masses for reaching the pH value.
3. Weighing the buffer salts.
4. Filling approximately 90% of the final volume of the solvent into a volumetric flask.
5. Adding the buffer salts.
6. Stirring the solution by means of a magnetic stirrer until the buffer salts have dissolved.
7. Checking the pH value and the temperature of the solution by means of a pH meter and a temperature sensor.
8. Adding acid solution or alkaline solution by means of a pipette, until the desired pH value has been reached.
9. Removing the stir bar of the magnetic stirrer.
10. Adding the solvent up to the target volume.
11. Checking the pH value and the temperature by means of a pH meter and temperature sensor.
12. Transferring the buffer into a labeled storage vessel.
13. Cleaning all equipment.

It follows from the above reference laboratory process that even in this simply constructed test case, interactions of the laboratory user B with the laboratory environment 1 are required at several points. This relates, for example, to the weighing of the buffer salts in work step 3, the stirring of the solution by means of the magnetic stirrer in work step 6, the check of the pH value and the temperature in work step 7, or the like. In order to reduce the probability of errors in all these interactions to a minimum, the method according to the proposal provides information about the laboratory environment in a targeted manner.

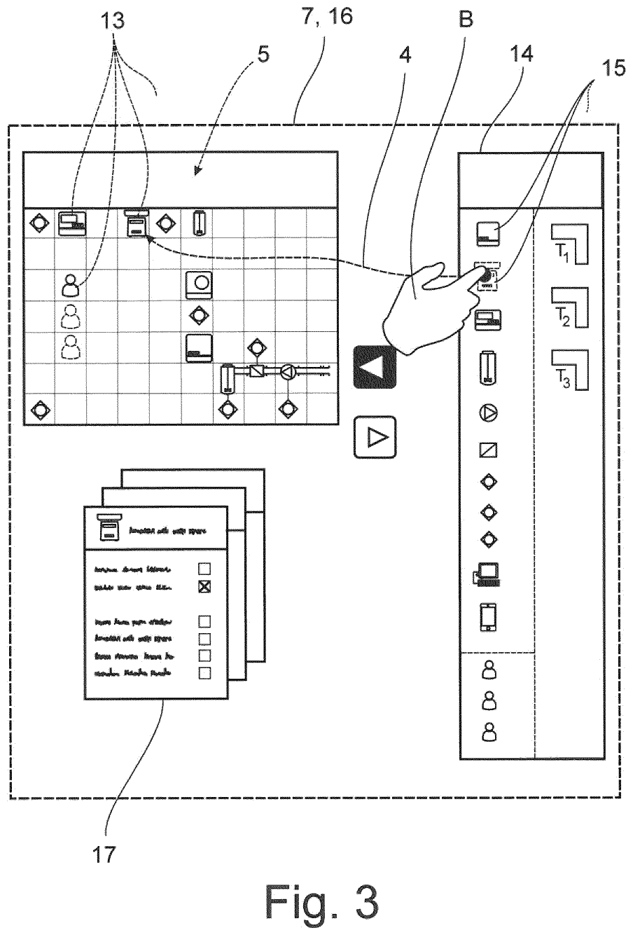

According to the proposal, it is initially provided that, by means of the assistance system 2, in a configuration step 4, the laboratory environment 1 is mapped in an interchangeable laboratory data model 5 using data technology. The interchangeabilty of the laboratory data model 5 is of particular importance for enabling the assistance system 2 to be easily adapted to new laboratory environments 1. For this purpose, it is necessary for the laboratory data model 5 to be correspondingly designed to be manageable as such. This is precisely the case with an object-oriented structure of the laboratory data model 5, which is still to be explained. The above configuration step 4 may be provided in a user-guided manner, as indicated in FIG. 3 and explained below. Alternatively or in addition, it may be provided that the configuration step 4 runs automatically according to a configuration rule. This may be appropriate, for example, if the laboratory environment 1 is expanded to include a new laboratory entity 3.

According to the proposal, it is further provided that in an interaction step 6, user inputs 8, in particular speech inputs which are still to be explained, may be entered by means of the assistance system 2 via a user interface 7, and predetermined user commands 9 which are matched to the laboratory data model 5 are derived from the user inputs 8. The derivation of the user commands 9 is matched to the laboratory data model 5 in such a way that, for example, only such user commands are derived which can also be implemented using the available laboratory entities 3 of the laboratory environment 1. This is associated with a reduction in the probability of errors in the interaction of the laboratory user B with the laboratory environment 1.

However, the method according to the proposal goes one step further. According to the proposal, in an implementation step 10, the derived user commands are implemented by means of the assistance system 2, based on the laboratory data model 5. This means that when implementing a previously derived user command 9, the laboratory data model 5 is accessed in order to be able to carry out the implementation in a manner which is tailored to the respective laboratory environment 1 to the greatest possible extent. This relates, for example, to an actuation of a laboratory device which is as user-friendly as possible, or the creation of documentation in which relevant state information about the laboratory environment 1 is automatically included.

Numerous possibilities are conceivable for implementing the method according to the proposal. Here, it is provided that the assistance system 2 is implemented at least partially in a cloud-based manner. Alternatively or in addition, the assistance system 2 runs at least partially as an app on a smart device 11. In particular, it is advantageous if the user interface 7 is provided by such a smart device 11. Other variants for the implementation of the user interface 7 are conceivable.

The interaction step 6 and/or the implementation step 10 is/are carried out not only based on the laboratory data model 5, but also additionally as a function of at least one piece of state information 12. The state information 12 represents the current situation prevailing in the laboratory environment 1, which relates, for example, to the location of the laboratory user B or the laboratory device values provided by the laboratory devices. In detail, the state information 12 is thus, for example, the location of the laboratory user B in the laboratory environment 1, and/or the laboratory entities 3 located in a predetermined vicinity of the laboratory user B, and/or the state of a laboratory entity 3 which is relevant to the process.

If the state information 12 is laboratory device values, it can be the case that in a readout step, laboratory device values, in particular measured values, of laboratory entities 3, in particular laboratory devices, can be received via a device interface for determining the state of a laboratory entity 3, by means of the assistance system 2.

However, the above state information 12 may also be a piece of information from any data source associated with the laboratory environment 1. This relates, for example, to a camera which documents the execution of a work step of the laboratory process.

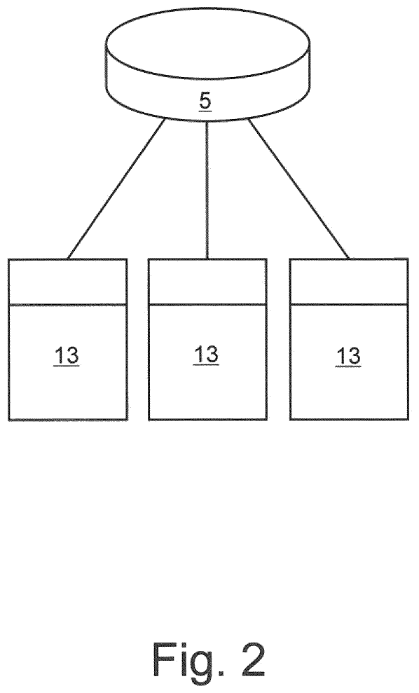

The basic structure of the laboratory data model 5 can be seen in the depiction according to FIG. 2. Accordingly, the laboratory data model 5 comprises a number of laboratory data objects 13 which respectively map a laboratory entity 3 of the laboratory environment 1 using data technology. In some embodiments, the laboratory data objects 13 are stored in the respective laboratory entity and/or can be read out from the respective laboratory entity. In the simplest case, a data link is stored in the respective laboratory entity 3, via which the actual laboratory data object can be downloaded from a remote server, in particular a cloud server. In this respect, the term “can be read out” is to be understood broadly.

Here, the laboratory data model 5 is structured in an object-oriented manner, based on predetermined data classes of laboratory entities 3, in such a way that the laboratory data objects 13 are respectively parameterized instances of a respective laboratory entity data class. The laboratory entity data classes may summarize different types of laboratory entities 3. For example, the laboratory entities 3 representing a laboratory device are associated with a laboratory entity data class “laboratory device.” Alternatively or in addition, for example, it is provided that the laboratory entities 3 representing a sample holder are associated with a laboratory entity data class “sample holder.” Alternatively or in addition, it may be provided that the laboratory entities 3 representing a laboratory user B are associated with a laboratory entity data class “laboratory user.”

By classifying the laboratory entities 3, it is possible to create the laboratory data objects 13 of the laboratory data model 5 in a particularly simple manner. The reason for this is that a laboratory entity data class comprises a set of at least partially parameterizable properties which is always associated with the laboratory data object 13 when creating the relevant laboratory data object 13 of the relevant laboratory entity data class.

The properties which are associated with a laboratory entity data class may comprise attributes such as size ratios, inputs/outputs, or the like, as well as methods such as reading out measured values or the like. Some of the properties may also be encapsulated, so that the probability of errors when creating laboratory data objects 13 can be further reduced.

The object-oriented structure of the laboratory data model 5 is particularly advantageous with respect to the interchangeabilty of the laboratory data model 5. In some embodiments, an instance of the object-oriented laboratory data model 5 is stored as such in the assistance system, thus correspondingly simplifying the replacement of the entire laboratory data model 5 as a unit.

A particularly simple variant for carrying out the configuration step 4 can be seen in the depiction according to FIG. 3. Here, it is provided that in the configuration step 4, the laboratory data model 5 is compiled from its library 14 of library objects 15 via the user interface 7. This is achieved here by associating library objects 15 with the laboratory data model 5 on the user side in a graphical configuration screen 16, here by using drag & drop. In the object-oriented structuring of the laboratory data model 5, the library objects 15 in some embodiments respectively represent a laboratory entity data class, so that the user-side association via the configuration screen 16 is accompanied by the creation of an instance of the relevant laboratory entity data class.

In the configuration screen 16 depicted in FIG. 3, the laboratory entities 3 are respectively depicted as graphical icons. On the right-hand side, the configuration screen 16 comprises a graphical representation of the library 14 with the library objects 15, which may be transferred by the user to the laboratory data model 5, which is also graphically depicted. Here, this is done using drag & drop, as mentioned above. The parameterization of the laboratory data objects 13 can be provided via the input fields 17, which are arranged below the representation of the laboratory data model 5.

Interesting in the depiction of the library 14 according to FIG. 3 is the fact that the icons depicted in the right-hand column of the library 14 respectively represent a so-called template T₁, T₂, T₃ which, are preconfigured laboratory environments 1 or parts of preconfigured laboratory environments 1. Thus, it is possible to configure recurring laboratory environments 1 in a similar form, in a particularly simple manner.

In order to ensure optimal functioning of the method according to the proposal, it can be the case that the laboratory data model 5 is adapted, in particular continuously, to the respectively current laboratory environment 1, and that the interaction step 6 and the implementation step 10 always access the respectively current laboratory data model 5. Thus, in principle, it is possible to provide that the laboratory data model 5 is updated automatically, as mentioned above, in particular in response to a change in the laboratory environment 1. In the exemplary embodiment which is depicted, the laboratory data model 5 is adapted to the respective current laboratory environment 1 by means of a user input 8, here via the configuration screen 16. A combination of both variants of the updating of the laboratory data model 5 is conceivable.

The advantageousness of the object-oriented structure of the laboratory data model 5 may be demonstrated particularly well with the aid of the reference laboratory process. The reference laboratory process requires at least some laboratory entities 3 in the laboratory environment 1, namely, an analytical balance (work step 3), a magnetic stirrer (work step 6), a pH meter and a temperature sensor (work steps 7 and 11), and a pipette (work step 8). Accordingly, the laboratory data model 5 must comprise at least the laboratory data objects 13 associated with these laboratory entities 3. It can be seen from the depiction according to FIG. 3 that it is possible to carry out the configuration step 4 for the reference laboratory process with a few user inputs 8, thus resulting in the laboratory data model 5 depicted by way of example in FIG. 2.

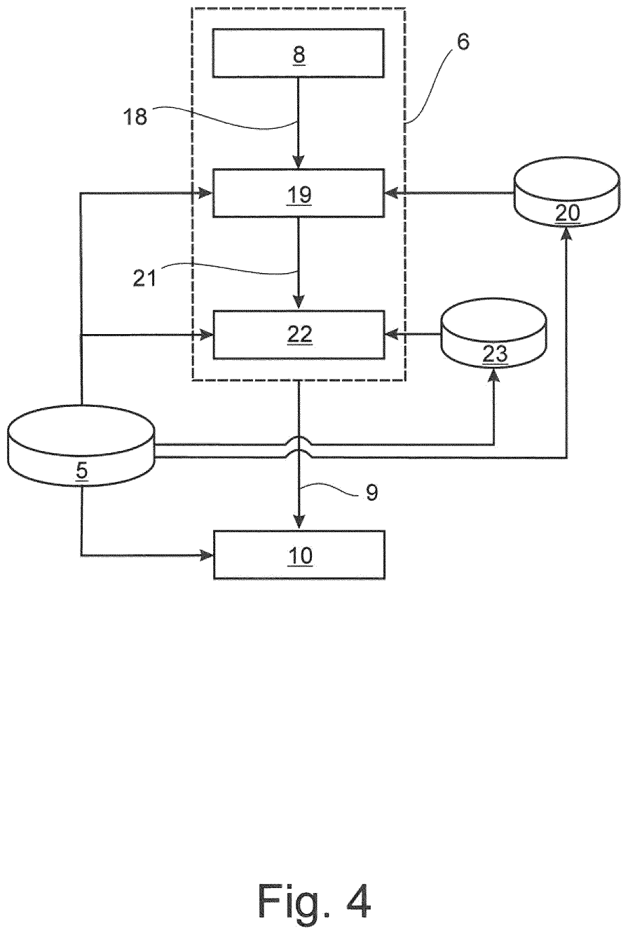

In some embodiments, the user inputs 8 are at least partially speech inputs, wherein the method according to the proposal provides a special system for speech processing. This speech processing system may be seen in principle from the depiction and in FIG. 4. Accordingly, in the interaction step 6, audio signals 18 can be detected via the user interface 7, wherein in a speech recognition step 19, a structured text 21 is generated from the audio signals 18, based on a language model 20.

Further, the speech recognition step 19 can be carried out as a function of the laboratory data model 5 and/or as a function of the aforementioned state information 12. This means that the speech recognition step 19 is carried out in different ways depending on the laboratory data model 5. In detail, this can mean that the language model 20 underlying the speech recognition step 19 is selected or modified as a function of the laboratory data model 5 and/or as a function of an above piece of state information 12. For example, it may be provided that the vocabulary underlying the language model 20 is attached to the laboratory data model 5. Using the example of the reference laboratory process, this means that the language model 20 does not have to comprise the portion of the vocabulary which, for example, is directed to a unit for liquid handling, since a unit for liquid handling is not included in the laboratory data model 5. This significantly reduces the complexity of the speech recognition. If the language model 20 is to be selected or modified as a function of the aforementioned state information 12, it can be the case that the language model 20 is tailored to the respectively existing and/or process-relevant laboratory entities 3.

Alternatively or in addition, at least a portion of the language model 20 may be included in the laboratory data model 5, in particular in the laboratory data objects 13. In principle, it is also possible that at least a portion of the language model 20 is read out from the relevant laboratory entity 3, in particular from the corresponding laboratory device, in the above sense.

FIG. 4 further shows that the interaction step 6 comprises a semantic analysis step 22, in which a semantic analysis of the structured text 21 generated in the speech recognition step 19 is carried out based on a semantic model 23, wherein in the semantic analysis, the respective user command 9 is derived from the structured text 21.

An above semantics analysis step 22 is known in principle, as is the aforementioned speech recognition step 19. However, the semantics analysis step 22 is again carried out as a function of the laboratory data model 5. This can mean that such user commands which have no relation to the actually existing laboratory entities 3 of the laboratory environment 1 are not taken into consideration in the semantic analysis step 22. In detail, it is accordingly provided that the semantic model 23 underlying the semantics analysis step 22 is selected or modified as a function of the laboratory data model 5 and/or as a function of an aforementioned piece of state information 12. Alternatively or additionally, it may also be provided here that at least a portion of the semantic model 23 is included in the laboratory data model 5, here, in the laboratory data objects 13.

Using the example of work step 3 of the reference laboratory process, a speech input by the laboratory user B could include the pronunciation of the command “weigh buffer salts.” Due to the fact that the language model 20, as mentioned above, is tailored to the laboratory environment 1 which is reduced in the reference laboratory process, no problems result in the speech recognition. The same applies to the performance of the semantic analysis step 22, since the few laboratory entities 3 present in the laboratory environment 1 allow a small number of predetermined user commands. Therefore, the semantics analysis step 22 is also easy to implement and is associated with a low probability of errors.

As mentioned above, the implementation of the respective user command 9 is also provided as a function of the laboratory data model 5. This is based on the knowledge that only an unambiguous implementation of the user command 9 which is tailored to the respective laboratory entity 3 ensures error-free processing. In detail, in this sense, it is proposed that an implementation rule is provided for each user command 9, according to which the respective user command 9 is implemented, and which, in some embodiments, is at least partially included in the associated laboratory data object 13. A replacement of the relevant laboratory entity 3 thus automatically results in a corresponding adaptation of the implementation rule.

In various embodiments, it is possible to read out at least a portion of the implementation rule from the associated laboratory entity 3, in particular from the associated laboratory device, in the above sense. This makes the above automatic adjustment particularly easy to implement.

The implementation rule may be implemented in very different ways. In some embodiments, a control sequence for implementing the user command 9 and the laboratory entities 3 involved in the implementation of the user command 9 are included in the implementation rule. Using the example of the work step 3 of the reference laboratory process, the implementation rule for the user command “weigh buffer salts” comprises a control sequence for the associated analytical balance, so that the analytical balance starts a measurement cycle and outputs the corresponding measured value in a display.

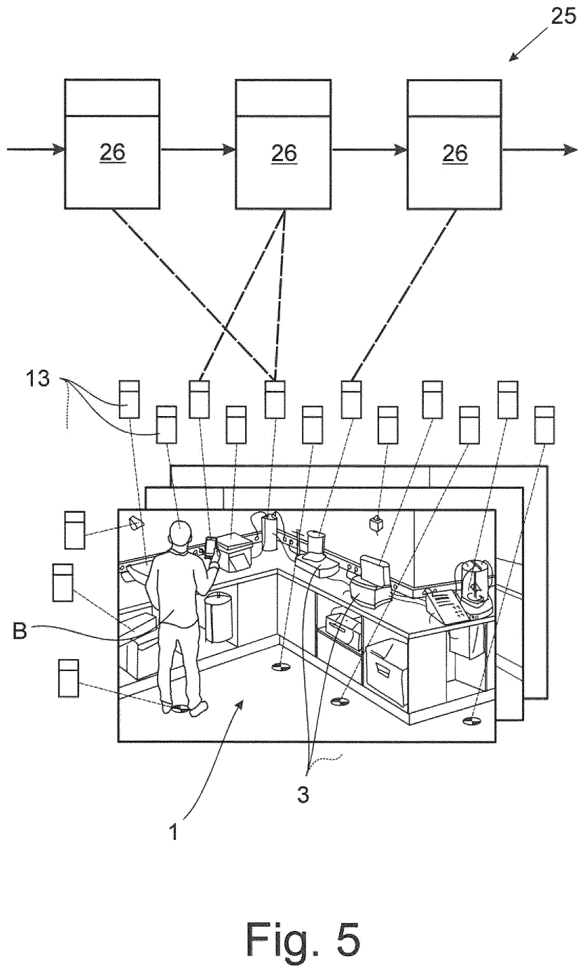

In some embodiments, at least one predetermined user command 9 relates to the documentation of the actual laboratory process. In this context, laboratory documentation 24 of the actual laboratory process in the laboratory environment 1 can be defined, wherein a predetermined user command 9 is the updating of the laboratory documentation 24. In some embodiments, the updating of the laboratory documentation 24 is event-based, wherein, an event triggering the updating can be a user input 8. In some embodiments, a laboratory documentation data structure 25 indicated in FIG. 5 for mapping the actual laboratory process in the laboratory environment 1 is associated with the laboratory documentation 24, wherein the laboratory documentation data structure 25 comprises a number of laboratory documentation data objects 26 which respectively map work progress in the laboratory process.

The work progress mapped by the laboratory documentation data objects 26 may be any type of event. Here, these events are user-related events and/or device-related events.

FIG. 5 shows that at least one laboratory data object 13 is associated with at least a portion of the laboratory documentation data objects 26 in each case. This means that, in addition to the actual laboratory process in the narrow sense, the laboratory documentation data structure 25 also includes information about the laboratory entities 3 relevant to the laboratory process. As a result, the laboratory documentation data structure 25 is a data structure which is at least partially configured in an object-oriented manner and which allows structured access to all data which are relevant to the laboratory process.

Alternatively or in addition, it may be provided that the updating of the laboratory documentation data structure 25 is carried out based on an aforementioned piece of state information 12. It may be provided that at least a portion of the aforementioned received laboratory device values are associated with at least a portion of the laboratory documentation data objects 26. Thus, device values may be entered automatically into the laboratory documentation 24 without having to be triggered by the laboratory user.

Using the example of the reference laboratory process, this means that the laboratory documentation data structure 25 comprises not only laboratory documentation data objects 26 which correspond to the total of 13 work steps, but also the laboratory data objects 13 which map the laboratory entities 3 of the analytical balance, the magnetic stirrer, the pH meter having a temperature sensor, the pipette, and the pump. Furthermore, it can be the case that, for example, the measured value of the weight of the buffer salts determined in work step 3 is received by means of the assistance system 2 and associated with the relevant laboratory documentation data object 26. Thus, the relevant measured value is stored in a logically structured manner without the laboratory user B having to give any organizational instructions.

The depiction according to FIG. 1 indicates that the laboratory documentation 24 comprises not only the laboratory documentation data structure 25, but also the audio data 18 for the respective speech inputs and the structured text 21 determined within the scope of the speech recognition step 19. Thus, there are effectively three types of descriptions for the laboratory documentation. The resulting redundancy results in a particularly high level of reliability when determining the actual laboratory process based on laboratory documentation 24.

The assistance system 2 according to the proposal may provide a plurality of further predetermined user commands 9. For example, a predetermined user command 9 may be the actuation of laboratory entities 3, in particular laboratory devices, wherein the actuation of the laboratory entities 3 is carried out in response to a corresponding user command 9 according to the associated implementation rule, based on the laboratory data model 5, in particular based on the corresponding laboratory data object 13. The relevant laboratory data object 13 includes, for example, a communication protocol for communication with the relevant laboratory entity 3, in particular the relevant laboratory device. As a result, communication with the relevant laboratory device is always ensured, even if the laboratory device was exchanged when updating the laboratory data model 5.

Alternatively or in addition, it may be provided that a predetermined user command 9 is the request for consumables, wherein the request for consumables is carried out in response to a corresponding user command 9 according to the associated implementation rule, based on the laboratory data model 5, in particular based on the corresponding laboratory data object 13. Specifically, this may be the consumable which the laboratory entity 3 needs for its operation. Using the example of a laboratory entity 3 in the form of a single-use bioreactor, the consumable to be requested may be a single-use reactor bag which is suitable for the bioreactor. An explicit request on the part of the laboratory user B is superfluous according to this variant of the method according to the proposal.

Further alternatively or in addition, it may be provided that a predetermined user command 9 comprises a translation step in which the documentation data structure 25 is translated into a selected national language, in particular, into a natural-language form, based on a translation rule. Here, it become clear that the laboratory documentation data structure 25 effectively provides a metadata format which is independent of a national language and can therefore be machine-translated into any national language.

The above independence of the laboratory documentation 24 according to the proposal from the respective national language is particularly advantageous with respect to the cooperation of laboratory environments 1 in which communication takes place using a different national language, a different dialect, a different laboratory jargon, or the like. The laboratory documentation data structure 25 according to the proposal is identical for all these laboratory environments 1, and allows, by means of the above translation step, simple machine translation into the respective national language used, the respective dialect used, and the respective laboratory jargon used.

It should still be pointed out that the independence from a national language relates not only to the laboratory documentation data structure 25, but also to the laboratory data model 5. All embodiments in this regard apply corresponding to the laboratory data model 5.

In view of the continuous availability of the laboratory data model 5 in every phase of the laboratory process, it is possible for the user inputs 8 to undergo a plausibility check with comparatively little effort. For this purpose, it is proposed that, in a plausibility step, the user input 8 is checked according to a plausibility rule in terms of plausibility with respect to the laboratory environment 1. In the event of an implausible user input 8, a warning message can be issued via the user interface 7.

It has already been explained that the assistance system 2 according to the proposal may be implemented in a cloud-based manner, and thus has an Internet connection to a cloud server 27. Following this concept, in principle, several spatially separated sub-laboratory environments may be provided, which are associated with the assistance system 2 in a manner according to the proposal via an Internet connection, in particular via a cloud server 27.

Finally, it should also be pointed out that the laboratory data model 5 may in principle also comprise a mapping of the laboratory process itself In this case, any laboratory actions which are associated with a laboratory process are also aforementioned laboratory entities 3 which are mapped by corresponding laboratory data objects 13. Consequently, the configuration screen 16 or a similar configuration screen may be used to create the laboratory processes based on the library objects 15 of a library 14. Here as well, it is conceivable that templates of predefined sub-processes can be selected in order to simplify the definition of a laboratory process.

According to a further teaching, an assistance system 2 for carrying out a method according to the proposal is provided. Reference may be made to all embodiments with respect to the method according to the proposal.

### LIST OF REFERENCE SIGNS

1 Laboratory environment
2 Assistance system
3 Laboratory entity
4 Configuration step
5 Laboratory data model
6 Interaction step
7 User interface
8 User input
9 User command
10 Implementation step
11 Smart device
12 State information
13 Laboratory data object
14 Library
15 Library object
16 Configuration screen
17 Input fields
18 Audio signal
19 Speech recognition step
20 Language model
21 Structured text
22 Semantic analysis step
23 Semantic model
24 Laboratory documentation
25 Laboratory documentation data structure
26 Laboratory documentation data object
27 Cloud server
B Laboratory user

1. A method for supporting laboratory processes in a laboratory environment by an assistance system,
wherein a number of laboratory entities are associated with the laboratory environment, and wherein, by the assistance system, in a configuration step, the laboratory environment is mapped in an interchangeable laboratory data model using data technology,
wherein in an interaction step, user inputs may be entered by the assistance system via a user interface, and predetermined user commands which have been matched with the laboratory data model are derived from the user inputs,
wherein in an implementation step, the derived user commands are implemented by the assistance system, based on the laboratory data model.

2. The method as claimed in claim 1, wherein the assistance system is implemented at least partially in a cloud-based manner, and/or in that the assistance system runs at least partially as an app on a smart device.

3. The method as claimed in claim 1, wherein the interaction step and/or the implementation step is/are also carried out as a function of at least one piece of state information, wherein the state information relates to the location of the laboratory user in the laboratory environment, and/or to the laboratory entities located in a predefined vicinity of the laboratory user, and/or to the state of an laboratory entity.

4. The method as claimed in claim 3, wherein in a readout step, laboratory device values of laboratory entities, can be received via a device interface for determining the state of a laboratory entity by the assistance system.

5. The method as claimed in claim 1, wherein the laboratory data model comprises a number of laboratory data objects) which respectively map a laboratory entity of the laboratory environment using data technology, wherein the laboratory data objects are stored in the respective laboratory entity and/or can be read out of the respective laboratory entity.

6. The method as claimed in claim 1, wherein the laboratory data model is structured in an object-oriented manner, based on predetermined data classes of laboratory entities, in such a way that the laboratory data objects are respectively parameterized instances of a respective laboratory entity data class, wherein laboratory entities representing a laboratory device are associated with a laboratory entity data class “laboratory device,” and/or in that laboratory entities representing a sample holder are associated with a laboratory entity data class “sample holder,” and/or in that the laboratory entities representing a laboratory user are associated with a laboratory entity data class “laboratory user.”

7. The method as claimed in claim 6, wherein an instance of the object-oriented laboratory data model is interchangeably stored as such in the assistance system.

8. The method as claimed in claim 1, wherein in the configuration step, the laboratory data model is compiled from a library of library objects via the user interface.

9. The method as claimed in claim 8, wherein the library objects respectively represent a laboratory entity data class.

10. The method as claimed in claim 1, wherein the laboratory data model is adapted to the respective current laboratory environment, and in that the interaction step and the implementation step always access the respective current laboratory data model, wherein the laboratory data model is adapted to the respective current laboratory environment by a user input.

11. The method as claimed in claim 1, wherein in the interaction step, audio signals are detected via the user interface, and in that in a speech recognition step, a structured text is generated from the audio signals, based on a language model.

12. The method as claimed in claim 11, wherein the speech recognition step is carried out as a function of the laboratory data model and/or as a function of the state information, wherein the language model underlying the speech recognition step is selected or modified as a function of the laboratory data model and/or as a function of a piece of state information, and/or in that at least a portion of the language model is included in the laboratory data model, in particular in the laboratory data objects.

13. The method as claimed in claim 11, wherein the interaction step comprises a semantic analysis step, in which a semantic analysis of the structured text is carried out, based on a semantic model, and in that, in the semantic analysis, the respective user command is derived from the structured text.

14. The method as claimed in claim 13, wherein the semantic model underlying the semantics analysis step is selected or modified as a function of the laboratory data model and/or as a function of a piece of state information, and/or in that at least a portion of the semantic model is included in the laboratory data model, in particular in the laboratory data objects.

15. The method as claimed in claim 1, wherein an implementation rule is provided for each user command, according to which the respective command is implemented, wherein at least a portion of the implementation rule is included in the associated laboratory data object.

16. The method as claimed in claim 15, wherein a control sequence for implementing the user command and the laboratory entities involved in the implementation of the user command are included in the implementation rule.

17. The method as claimed in claim 1, wherein laboratory documentation of the actual laboratory process in the laboratory environment is defined, and in that a predetermined user command is the updating of the laboratory documentation wherein the updating of the laboratory documentation takes places in an event-based manner, wherein an event triggering the updating is a user input.

18. The method as claimed in claim 17, wherein a laboratory documentation data structure for mapping the actual laboratory process in the laboratory environment is associated with the laboratory documentation, and in that the laboratory documentation data structure comprises a number of laboratory documentation data objects which respectively map work progress in the laboratory process.

19. The method as claimed in claim 16, wherein the work progress mapped by the laboratory documentation data objects are user-related events and/or device-related events.

20. The method as claimed in claim 16, wherein at least one laboratory data object is associated with at least a portion of the laboratory documentation data objects in each case.

21. The method as claimed in claim 2, wherein the updating of the laboratory documentation data structure is carried out based on the piece of state information.

22. The method as claimed in claim 3, wherein at least a portion of the received laboratory values are associated with at least a portion of the laboratory documentation data objects.

23. The method as claimed in claim 1, wherein a predetermined user command is the actuation of laboratory entities, in particular laboratory devices, and in that the actuation of the laboratory entities is carried out in response to a corresponding user command based on the laboratory data model.

24. The method as claimed in claim 1, wherein a predetermined user command is the request for consumables, and in that the request for consumables is carried out in response to a corresponding user command based on the laboratory data model.

25. The method as claimed in claim 1, wherein a predetermined user command comprises a translation step in which the documentation data structure is translated into a selected national language based on the translation rule.

26. The method as claimed in claim 1, wherein in a plausibility step, the user input is checked according to a plausibility rule in terms of plausibility with respect to the laboratory environment.

27. An assistance system for carrying out a method as claimed in claim 1.
